(19) United States
(12) Patent Application Publication
(10) Pub. No.: US 2015/0273015 A1
(43) Pub. Date: Oct. 1, 2015

(54) TREATMENTS OF HEPATITIS C VIRUS INFECTION

(71) Applicant: Claudio Avila, Basel (CH)

(72) Inventor: Claudio Avila, Basel (CH)

(73) Assignee: Novartis AG, Basel (CH)

(21) Appl. No.: 14/735,962

(22) Filed: Jun. 10, 2015

Related U.S. Application Data
(63) Continuation of application No. 13/990,097, filed on May 29, 2013, now abandoned.
(60) Provisional application No. 61/418,137, filed on Nov. 30, 2010.

(51) Int. Cl. A61K 38/13; A61K 38/21; A61K 31/7056; A61K 31/549

(52) U.S. Cl. CPC A61K 38/13; A61K 31/549; A61K 38/21; A61K 31/7056; A61K 38/212

(57) ABSTRACT

The invention concerns the use of cyclophilin inhibitors in the treatment of Hepatitis C virus infection.

The present disclosure relates to a non-immunosuppressive cyclosporin which binds to cyclophilin, which are cyclophilin inhibitors, in particular to their pharmaceutical use of in the treatment of Hepatitis C virus infection.

The cyclosporins comprise a class of structurally distinctive, cyclic, poly-N-methylated undecapeptides, commonly possessing pharmacological, in particular immunosuppressive, or anti-inflammatory activity. The first of the cyclosporins to be isolated was the naturally occurring fungal metabolite Ciclosporin or Cyclosporine, also known as cyclosporin A (CsA).

Cyclosporins which bind strongly to cyclophilin but are not immunosuppressive have been identified. PCT/EP 2004/009804, WO 2005/021028, or WO 2006/071619 disclose non-immunosuppressive cyclosporins which bind to cyclophilin have also been found to have an inhibitory effect on Hepatitis C virus (HCV). WO 2006/038088, incorporated herein by reference in its entirety, describes methods and compositions for the use of alisporivir in the treatment of HCV. Alisporivir (Debio-025 or DEB025 or DEB) is a cyclophilin (Cyp) inhibitor and its mode of action as an anti-HCV agent is via inhibition of host proteins, in particular of cyclophilin A, that are directly involved in HCV replication.

Hepatitis C virus (HCV) is an enveloped single stranded (+) RNA virus that belongs to the separate genus Hepacivirus of the family Flaviviridae. HCV causes acute and chronic liver disease, including chronic hepatitis, cirrhosis, and hepatocellular carcinoma. Worldwide more than 170 million people are chronically infected with HCV and are thus at increased risk of developing serious life-threatening liver disease.

The current standard of care in HCV patients consists of a combination of interferon and ribavirin. Treatment duration and ribavirin dose depend on the genotype treated. Sustained viral response (SVR) in patients with genotypes 2 and 3 after standard of care treatment reaches 80-90%, but only 40-50% in patients with genotype 1. Moreover, a slower response has been indicated as an important parameter to determine relapsers. Furthermore, side effects are significant and include myalgia, arthralgia, headache, fever, severe depression, leucopenia and haemolytic anaemia.

As a result, there is currently a large proportion of chronic HCV infected patients that are in high need for new treatment modalities that would allow them to achieve SVR and halt the further evolution of their chronic liver disease. Persistent infection by HCV, which has been identified as the major causative agent of non-A, non-B hepatitis has been considered closely related to liver diseases such as chronic hepatitis, liver cirrhosis or hepatocellular carcinoma. The development of these liver diseases is a major public health problem.

Despite the positive indications in the art of the use of CsA and non-immunosuppressive cyclosporine in treatment of HCV, there is a significant class of HCV patients that remains refractory to the current standard of care therapies. Thus, despite existing therapies, there remains a significant need for methods and compositions for the treatment of HCV.

We have found out that cyclophilin inhibitors, in particular alisporivir, can be used effectively in the treatment of HCV. In particular, we have found that satisfactory treatment results of Hepatitis C virus, genotype 1, 2, 3 or 4 infection can be obtained when using alisporivir twice per day.

The distinctive feature of alisporivir to interact with host rather than viral targets provides at least two advantages of clinical significance, like for example a high barrier for emergence of specific drug resistance mutations and efficacy in all HCV genotypes.

Accordingly, the present invention provides new anti-HCV treatments using alisporivir, in particular methods of treating hepatitis C virus, all genotypes, infection in a patient comprising administering to the patient alisporivir, in an amount of about 400 to about 600 mg twice per day.

The invention further provides alisporivir for use in the treatment or prevention of Hepatitis C virus infections or HCV induced disorders in a patient.

### SUMMARY OF THE DISCLOSURE

Further, the following is described:

1.1 A method for preventing or treating Hepatitis C infections or HCV induced disorders in a patient, comprising administering to said patient alisporivir in an amount of about 400 to about 600 mg twice per day.

1.2 A method for inhibiting HCV replication in a patient, comprising administering alisporivir in an amount of about 400 to about 600 mg twice per day.

1.3 A method for preventing or delaying the recurrence of HCV infection in a transplant recipient, comprising administering to said recipient alisporivir in an amount of about 400 to about 600 mg twice per day.

2. Use of alisporivir in the preparation of a pharmaceutical composition for use in any method as defined above.

3. Use of alisporivir in the preparation of a medicament for use in any method as defined above.

4. A pharmaceutical composition for use in any method as defined above, comprising alisporivir, together with one or more pharmaceutically acceptable diluents or carriers therefore.

5. A therapeutic regimen comprising administering alisporivir in an amount of about 400 to about 600 mg twice per day and wherein alisporivir is administered in combination with standard of care or in combination with one or more direct acting antiviral agents.

6. A package comprising the pharmaceutical composition comprising alisporivir as defined above, in combination with instructions to administer said composition in an amount of about 400 to about 600 mg twice per day.

7. A kit for the treatment of chronic hepatitis C infection.

Also contemplated herein are methods of reducing the HCV RNA in a patient comprising administering to the patient: alisporivir, an interferon; and a ribavirin in which alisporivir is to be administered in an amount of about 400 or about 600 mg twice per day.

Additional embodiments of the present invention relate to methods of treating hepatitis C genotype 1 infections in a patient that is resistant, or non-responder to standard of care therapy for HCV treatment comprising administering to the patient: alisporivir in combination with standard of care, wherein alisporivir is to be administered in an amount of about 400 to about 600 mg twice per day.

Also contemplated herein is a pharmaceutical combination comprising a first pharmaceutically acceptable formulation comprising alisporivir, a second pharmaceutically acceptable formulation comprising an interferon and a third pharmaceutically acceptable formulation comprising ribavirin, wherein the first, second and third formulations are packaged in a kit for the treatment of chronic hepatitis C infection.

Also contemplated herein is a pharmaceutical combination comprising a first pharmaceutically acceptable formulation comprising alisporivir, a second pharmaceutically acceptable formulation comprising a direct acting antiviral agent, wherein the first and second formulations are packaged in a kit for the treatment of chronic hepatitis C infection.

### DETAILED DESCRIPTION OF THE DISCLOSURE

In the above embodiments and throughout this specification, the standard of care treatment is a treatment that is used to treat Hepatitis C infections. The currently used standard of care treatment involves administration of interferon, in particular pegylated interferon in combination with ribavirin.

In the present application, the term “non-responder” is intended to mean a patient or subject who is a non-responder to standard of care treatment for HCV. More specifically, a non-responder to standard of care patient is a patient who has not responded to treatment with standard of care given over a 12 week treatment period. The non-responder to standard of care includes the following subsets of patients—null responders and partial responders.

Typically, a patient who has a “null response” may, for example, be defined as one in whom the HCV-RNA reduction is observed to be less than about 2 log10 IU/mL , e.g. less than 2 log10 IU/mL, after 12 weeks of treatment with standard of care.

A patient that has a “partial” response or partial responder is one in whom the HCV-RNA reduction of more than about 2 log10 IU/mL , e.g. less than 2 log10 IU/mL, is observed after 12 weeks of treatment with standard of care but the HCV-RNA is still detectable at the end of treatment.

As used herein, “microgram/kilogram” means microgram drug per kilogram body weight of the mammal—including man—to be treated.

By “therapeutic regimen” is meant the pattern of treatment of an illness, e.g., the pattern of dosing used during HCV therapy. A therapeutic regimen may include an induction regimen and a maintenance regimen.

As used herein, the term “about”, unless the context dictates otherwise, is used to mean a range of + or −10%.

As used herein “up to 12, 24, 48 or 72 weeks” refers to the treatment duration and is intended to mean for about 12 weeks, about 24 weeks, about 48 weeks, or about 72 weeks, respectively. It will be understood that therapy need not end at exactly the 12, 24, 48 or 72 week time period. For example, therapy may end a day or a few days before the 24 week period, and still be an equivalent within the scope and spirit of the current disclosure.

As used herein “twice per day” or BID means twice in any period of about 24 hour period; “once per day” or QD means once in any period of about 24 hour period; “once per week” is used to mean once in any period of about seven days.

HCV RNA levels can be measured using commercially available methods. As used herein, LOD means limit of detection and LOQ means limit of quantification of HCV RNA levels. For example, when using the COBAS® TaqMan® HCV Test, v2.0 (Roche Diagnostics) for assessment of HCV RNA levels, LOQ of 25 IU/ml (1.398 log10) and LOD of 10 IU/ml (1 log10) have been reported.

In the present invention, an interferon may be pegylated or non-pegylated and may include interferons such as: Intron-A®, interferon alfa-2b (Schering Corporation, Kenilworth, NJ); PEG-Intron®, peginteferon alfa-2b (Schering Corporation, Kenilworth, N.J.); Roferon®, recombinant interferon alfa-2a (Hoffmann-La Roche, Nutley, N.J.); Pegasys®, peginterferon alfa-2a (Hoffmann-La Roche, Nutley, N.J.); Berefor®, interferon alfa 2 available (Boehringer Ingelheim Pharmaceutical, Inc., Ridgefield, Conn.); Sumiferon®, a purified blend of natural alpha interferons (Sumitomo, Japan); Wellferon®, lymphoblastoid interferon alpha n1 (GlaxoSmithKline); Infergen®, consensus alpha interferon (InterMune Pharmaceuticals, Inc., Brisbane, Calif. and Amgen, Inc., Newbury Park, Calif.); Alferon®, a mixture of natural alpha interferons (Interferon Sciences, and Purdue Frederick Co., Conn.); Viraferon®; and combinations of these interferons.

Conjugated interferons that may be used include, for example, Albuferon (Human Genome Science) which is conjugated to human albumin. Interferon conjugated to a water-soluble polymer or polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Interferon-polymer conjugates are described in U.S. Pat. No. 4,766,106, U.S. Pat. No. 4,917,888, EPA 0 236 987, EPA 0 510 356 and WO 95/13090. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Other forms of interferons include interferon beta, gamma, tau and omega, such as Rebif (Interferon beta 1a) by Serono, Omniferon (natural interferon) by Viragen, or Omega Interferon by Boehringer Ingelheim. Oral interferons such as oral interferon alpha by Amarillo Biosciences.

Additional examples of interferons that may be used include pegylated interferon alpha, for example pegylated interferon α-2a, pegylated interferon α-2b, pegylated consensus interferon or pegylated purified interferon-α product. Pegylated interferon α-2a is described in European Patent 593,868 (incorporated herein by reference in its entirety) and commercially available e.g. under the trade name PEGASYS® (Hoffmann-La Roche). Pegylated interferon-α-2b is described, e.g. in European Patent 975,369 (incorporated herein by reference in its entirety) and commercially available e.g. under the trade name PEG-INTRON A® (Schering Plough). Pegylated consensus interferon is described in WO 96/11953 (incorporated herein by reference in its entirety).

In preferred embodiments, the interferon used in the methods of the invention is pegylated interferon. In other embodiments, the interferon is selected from the group consisting of interferon alpha-2a, Interferon alpha-2b, a consensus interferon, a purified interferon alpha product or a pegylated interferon alpha-2a, pegylated interferon alpha-2b, and pegylated consensus interferon, a mixture of natural alpha and combinations thereof.

Preferably the methods using interferon alpha use a pegylated interferon alpha-2b and the amount of pegylated interferon alpha-2b is from 0.5 to 2.0 micrograms/kilogram per week on a weekly, three times a week, every other day or daily basis.

In other embodiments, the interferon alpha is a pegylated interferon alpha-2a and the amount of pegylated interferon alpha-2a administered is from 20 to 250 micrograms/kilogram per week on a weekly, three times a week, every other day or daily basis. Preferably, the interferon peg-IFNa2a is administered at an amount of 180 ug once per week.

In specific embodiments, the exemplary interferon used in the methods herein is interferon selected from the group consisting of Intron-A®; PEG-intron®; Roferon®; Pegasys®; Berefor®; Sumiferon®; Wellferon®; Infergen®; Alferon®; Viraferon®; Albuferon® (Human Genome Science); Rebif; Omniferon; Omega and combinations thereof.

In some embodiments, ribavirin is administered at between about 800 to about 1200 mg per day, e.g., 1000 mg to 1200 mg per day. In some embodiments, ribavirin is administered based on the weight of the patient. In other embodiments, ribavirin is administered based on the HCV genotype of the patient.

In another embodiment, alisporivir may be administered with additional agents of the standard of care that promote the antiviral efficacy of the therapy treatment. Additional agents that promote the antiviral efficacy of the therapy treatment, include polymerase inhibitors, protease inhibitors, substrate-based protease inhibitors of HCV NS3-4A serine protease, non-substrate-based NS3 protease inhibitors; phenanthrenequinones, thiazolidines and benzanilides, nucleosides analogs, antisense molecules directed against HCV genome or any cellular component that is required for viral replication, vaccine or antibody-based approaches to HCV treatment.

Direct acting antiviral agents, is used herein to mean agents that interfere with specific steps in the hepatitis C virus (HCV) replication cycle. Such agents may be, e.g., ribavirin derivatives, protease inhibitors, polymerase inhibitors (e.g., nucleoside and non-nucleoside inhibitors), and cyclophillin inhibitors. Exemplary direct acting antiviral agents include: boceprevir, telaprevir, ABT-072, ABT-450, ABT-333 by Abbott, ACH1625 by Achillion, ANA598 by Anadys Pharmaceuticals, AZD-7295 by AstraZeneca, BI201335, BI207127 by Boehringer Ingelheim Pharma, BMS650032, BMS790052, BMS791325, BMS824383 by Bristol Myers Squibb, Clemizole by Eiger BioPharmacetucials, Filibuvir by Pfizer, GS9190 (Tegobuvir), GS9256 by Gilead, IDX375 by Idenix, INX-189 by Inhibitex, PSI-7851, PSI-938 by Pharmasset, PSI-7977, RG7128 by Pharmasset/Genethec, PPI-461 by Presidio RG7227 (Danoprevir) by InterMune/Genentech, SCH900518 (Narlaprevir), Vaniprevir by Merck, TMC435 by Medivir/Tibotec, VX-222, VX-759, VX-500, VX-916 by Vertex. In one embodiment, the present invention further provides alisporivir for use in combination with standard of care in treatment of a Hepatitis C virus infected patient, the alisporivir to be administered in an amount of about 400 to about 600 mg twice per day. In still another aspect, alisporivir is to be administered for up to 24, 48 or 72 weeks.

In one embodiment, the present invention further provides alisporivir for use in combination with interferon and ribavirin in treatment of a Hepatits C virus infected patient, the alisporivir being administered in an amount of about 400 mg twice per day for up to 72 weeks, preferably up to 48 weeks, most preferred up to 24 weeks.

In one embodiment, the present invention further provides alisporivir for use in combination with interferon and ribavirin in treatment of a Hepatitis C virus infected patient, the alisporivir being administered in an amount of about 400 mg twice per day for up to 24 weeks.

In one embodiment, the present invention further provides alisporivir for use in combination with standard of care, preferably with pegylated interferon alpha-2a and ribavirin in treatment of a Hepatitis C virus infected patient, the alisporivir is to be administered in an amount of about 400 to about 600 mg twice per day for up to 24 or 48 or 72 weeks. In still another aspect, the pegylated interferon alpha-2a and is administered in an amount of 180 micrograms once per week.

In one embodiment, the present invention further provides alisporivir for use in combination with pegylated interferon alpha-2a and ribavirin in treatment of a Hepatitis C virus infected patient, the alisporivir being administered in an amount of about 400 to about 600 mg twice per day for up to 24, 48 or 72 weeks. In still another aspect, the ribavirin is administered at between 800 mg to 1200 mg per day and the pegylated interferon alpha-2a is administered in an amount of 180 micrograms once per week.

In one aspect, the present invention further provides use of alisporivir in the manufacture of a medicament for treatment of a Hepatitis C virus infected patient wherein alisporivir is to be administered in an amount of about 400 to about 600 mg twice per day for up 24, 48 or 72 weeks and wherein alisporivir is administered in combination with interferon and ribavirin.

In one aspect, the present invention further provides use of alisporivir in the preparation of a pharmaceutical composition for treatment of a Hepatitis C virus infected patient characterized in that alisporivir is to be administered in an amount of about 400 to about 600 mg twice per day for up to 24, 48 or 72 weeks and wherein alisporivir is administered in combination with interferon and ribavirin. In one aspect, the present invention further provides a combination of alisporivir with standard of care, preferably with interferon and ribavirin for use in treatment of a Hepatitis C virus infected patient, wherein alisporivir is to be administered in an amount of about 400 to about 600 mg twice per day for up to 24, 48 or 72 weeks.

In one aspect, the present invention further provides a therapeutic regimen comprising administering alisporivir in an amount of about 400 to about 600 mg twice per day for up to 24, 48 or 72 weeks and wherein alisporivir is administered in combination with interferon and ribavirin.

In one aspect, the present invention further provides pharmaceutical compositions comprising alisporivir for uses as defined above. In still other aspects, the present invention provides a package comprising the pharmaceutical composition comprising alisporivir for uses as defined above in combination with instructions to administer said composition.

In exemplary embodiments, alisporivir is administered at a dosage of from about 400 to about 600 mg twice per day for up to 24, 48 or 72 weeks.

In exemplary embodiments, the treatment of the present invention involves administration of interferon alpha that is a pegylated interferon alpha-2a and the amount of pegylated interferon alpha-2a administered is from 20 to 250 micrograms per week on a weekly, three times a week, every other day or daily basis. The current approved dose is 180 micrograms per week. In other exemplary embodiments, the interferon alpha is a pegylated interferon alpha-2b and the amount of pegylated interferon alpha-2b is from 0.5 to 2.0 micrograms/kilogram per week on a weekly, three times a week, every other day or daily basis. Exemplary descriptions of such treatments are described in U.S. Pat. No. 7,115,578, incorporated herein by reference in its entirety.

An exemplary Peg-IFNα2a used in the treatment protocols described herein is Pegasys®. PEGASYS® is a pegylated form of IFNα2a and utilizes a 40 kDa branched PEG (polyethylene glycol) to provide sustained serum concentrations for a full week (168 hours). PEGASYS® is commercially available, presented as single use, pre-filled syringes containing 180 μg/0.5 mL peg-IFNα2a for S.C. injection. The standard package contains 1 syringe of 180 μg/0.5 mL.

In some embodiments, it may be desirable to modify the dose of Peg-IFNα2a. If dose modification is required for moderate to severe adverse reactions (clinical and/or laboratory), initial dose reduction from 180 to 135 μg is generally adequate (adjustment to the corresponding graduation mark on pre-filled syringe). However, in some cases, dose reduction to 90 μg may be needed. Following improvement, re-escalation of the dose may be considered.

In treatment described above effective dosages of the standard of care agents or of direct antiviral agents are administered in compositions, i.e. they may be administered together (i.e., simultaneously), but may also be administered separately or sequentially. In general, combination therapy is typically administered together, the rationale being that such simultaneous administration induces multiple simultaneous stresses on the virus. The specific dosages given will depend on absorption, inactivation and excretion rate of the drugs as well as other factors. It is to be noted that dosage values will also vary with the severity of the condition to be alleviated.

The terms “co-administration” or “combined administration” or “administered in combination with” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic or additive therapeutic effect, compared to a monotherapy applying only one of its pharmaceutically active ingredients or as compared to the current standard of care therapy. The treatment used in the methods described herein may be administered by any conventional route. One or more components may be administered parentally, e.g., in the form of injectable solutions or suspensions, or in the form of injectable deposit formulations. Preferably, alisporivir will be administered orally in the form of solutions or suspensions for drinking, tablets or capsules. Pharmaceutical compositions for oral administration comprising alisporivir typically further comprise one or more pharmaceutically acceptable carrier substances. Typically, these compositions are concentrated and need to be combined with an appropriate diluent, e.g., water, prior to administration. Pharmaceutical compositions for parenteral administration typically also include one or more excipients. Optional excipients include an isotonic agent, a buffer or other pH-controlling agent, and a preservative. These excipients may be added for maintenance of the composition and for the attainment of preferred ranges of pH (about 6.5-7.5) and osmolarity (about 300 mosm/L).

The efficacy of the therapy regimen may be monitored using standard protocols. Treatment may be followed by determinations of HCV in serum and measurement of serum ALT (alanine-aminotransferase) levels. For example, the patients may be assessed for the presence of IICV RNA in their plasma. HCV RNA (IU/mL) can be measured at regular intervals during the treatment, e.g., at Day 1 (pre-dose and 4, 8, and 12 hours post-dose) and pre-dose at Day 2, Day 3, Day 8, Day 15, Day 29, and at Week 12, Week 24, Week 36, Week 48, Week 72 (when applicable), and at follow up. In addition, the HCV strains in the patient can be sequenced and assessed for identification of mutations selecting for resistance.

As used herein, AUC refers to Area Under the Curve in [hr ng/mL] or area under the concentration vs time curve indicating the integrated quantity of analyte or drug (the serum concentration curve) after dosing; Cmax refers to the maximum concentration of the analyte or drug in [ng/mL] achieved after dosing; Cmin refers to the minimum concentration of the analyte or drug in [ng/mL] achieved after dosing

The endpoint of treatment is a virological response, i.e., the absence of HCV at the end of a treatment course, several months after initiation of treatment, or several months after completion of treatment. HCV in serum may be measured at the RNA level by methods such as quantitative RT-PCR or northern blots or at the protein level by enzyme immunoassay or enhanced chemiluminescence immunoassay of viral proteins. The endpoint may also include a determination of a serum ALT level in the normal range.

The virological response parameters are: rapid virologic response at treatment week 4 (RVR 4) defined by undetectable serum HCV-RNA at treatment week 4; early virological response (EVR), defined by at least 2 log10 IU/mL reduction in HCV-RNA compared to baseline (partial EVR) or undetectable serum HCV-RNA (complete EVR) at treatment week 12; sustained virological response (SVR24), defined as absence of HCV-RNA from serum by a sensitive Polymerase Chain Reaction (PCR) assay 24 weeks following end of therapy or the HCV RNA is undetectable (by LOD) 24 weeks after end of treatment; End of Treatment Response (ETR): HCV RNA undetectable (by LOD) at treatment end (completed or prematurely discontinued).

Exemplary therapeutic regimens are given in the Examples.

In one exemplary therapeutic regimen a subject in need of treatment is provided with pegylated interferon alfa 2a at a dose of 180 μg subcutaneously (S.C.) once weekly for 48 weeks in combination with ribavirin administered in an oral dosage of 800/1200 mg daily (weight based) for 24 weeks and 400 mg alisporivir orally twice daily for 24 weeks.

After a 4 week treatment period, based on patient response, the administration of alisporivir may be continued up to 48 or 72 weeks from the start of treatment at 600 or 800 mg once per day orally or preferably the dose of alisporivir is reduced to a lesser amount in a daily dose (e.g., 400 mg) or more preferably, the administration of alisporivir is discontinued. The treatment with pegylated interferon alfa 2a and ribavirin is preferably continued for up to 48 or 72 weeks from the initiation of treatment. For example the patient is administered 180 μg pegylated interferon alfa 2a S.C. orally once weekly and ribavirin administered in an oral dosage of 800/1200 mg daily (weight based).

### EXAMPLES

Combined with peg-IFNα2a, a dosing regimen of alisporivir 600 mg twice per day (BID) for one week followed by 600 mg once daily (QD) for a 48 week treatment duration has been demonstrated to be superior to peg-IFNα2a in a phase 2 study of genotype 1 treatment-naive patients based on rates of achievement of sustained virologic response (SVR).

In addition, study of the use of alisporivir in HCV Genotype 1 non responder patients addressed the utility of a 400 mg BID dose regimen versus the same total daily dose given once daily (800 mg QD) during the first week of treatment, both with peg-IFNα2a plus RBV, and the possible relationships between Cmax, AUC, or Cmin and antiviral effect at the end of that treatment period.

In this hard to treat patient population, the 400 mg BID dose provided greater decline in HCV RNA from baseline (−1.41 log10 IU/ml) than the 800 mg QD dose (−0.70 log10 IU/ml). Patients treated with 400 mg BID dose during the first week received 400 mg QD for the remaining 3 weeks of treatment (total 4 weeks treatment duration), while patients who received 800 mg QD dose regimen remained without modification during the entire 4-week treatment duration. The two treatment strategies (400 mg BID/400 mg QD and 800 mg QD) achieved similar declines in HCV RNA at the end of treatment (Day 29). The tolerability of 400 mg BID was favorable with similar rates of hyperbilirubinemia compared to 800 QD.

Consistent with this greater antiviral efficacy of the 400 mg BID dose during the first week of treatment, the median observed Cmin was also higher (175 ng/ml) than was achieved by the same daily dose given once a day, i.e. 800 mg QD dose (148 ng/ml).

Examination of the relationships between observed drug exposures (e.g., Cmin) and several virologic response measures using data from multiple clinical investigations including the phase 2 study suggest that a higher Cmin is associated with a higher likelihood of response. Consequently, a regimen of DEB025 400 mg BID is to be investigated as to whether the resulting Cmin maintained through the entire treatment duration (24 or 48 weeks) may be safe and achieve a better HCV RNA responses, translated into better RVR, EVR and eventually SVR results compared to peg-IFNα2a/RBV.

Efficacy of standard of care in chronic hepatitis C genotype 1 is unsatisfactory since only 40-50% achieve an SVR24 and up to 32% suffer relapse. Slow response has been implicated as an important parameter in this respect. The addition of a third drug with a different mechanism of action provide the advantage of reducing the number of non-responders to standard of care and reducing the time to HCV-RNA negativity in responders to standard of care for reduction of the rate of relapsers.

1. Compounds

Peg-IFNα2a is a pegylated form of interferon alfa 2a and utilizes 40 kDa branched PEG (polyethylene glycol) to provide sustained serum concentrations for a full week (168 hours). PEGASYS® is commercially available from Roche.

Ribavirin is a synthetic nucleoside analogue and is also commercially available, e.g., as COPEGUS® from Roche.

Alisporivir (Debio-025 or DEB025 or DEB) is a cyclophilin (Cyp) inhibitor and its mode of action as an anti-HCV agent is via inhibition of host proteins, in particular of cyclophilin A, that are directly involved in HCV replication.

2. Clinical Study

Patients receive for the entire treatment period duration dual combination treatment with: pegylated interferon-α (peg-IFNα2a) 180 μg s.c. once weekly plus ribavirin (RBV) 1000/1200 mg daily in 2 divided doses (morning/evening intake)

In addition to peg-IFNα2a/RBV, patients receive either alisporivir or placebo based on the treatment group they are randomized in:

A: Triple therapy with a response-guided treatment duration (see below) with peg-IFNα2a/RBV plus alisporivir 600 mg BID for one week followed by peg-IFNα2a/RBV plus alisporivir 600 mg QD for an additional 23 or 47 weeks based on week 4 HCV RNA results

B: Triple therapy with a response-guided treatment duration (see below) with peg-IFNα2a/RBV plus alisporivir 400 mg BID for 24 or 48 weeks based on week 4 HCV RNA results.

Response-guided treatment duration:

Patients with a viral load below the level of detection (LOD) at week 4 (<RVR4LOD) stop peg-IFNα2a/RBV and alisporivir study medications after 24 weeks.

Patients with a viral load at or above the level of detection (LOD) at week 4 (≧RVR4LOD) 1 complete the 48 weeks of peg-IFNα2a/RBV and alisporivir study treatment.

C: Fixed-duration triple therapy with peg-IFNα2a/RBV plus alisporivir 600 mg BID for one week followed by peg-IFNα2a/RBV plus alisporivir 600 mg QD for an additional 47 weeks

D: Active comparator arm with DEB025 placebo plus peg-IFNα2a/RBV for 48 weeks

Randomization of the patients will be stratified by HCV RNA level, IL28B polymorphism and BMI measured or defined at screening.

The IVRS/IWRS (interactive voice response system/Interactive Web Response System) will also be used to ensure that a maximum of 20% of patients with cirrhosis is randomized in each country. The randomization scheme for patients will be reviewed and approved by a member of the Biostatistics Quality Assurance Group.

Patients with concomitant total bilirubin levels >5×ULN (upper limit of normal) and one of the following:
ALT>ULN and 50% increased from baseline, or
ALT>5×ULN and increased from baseline
   interrupt alisporivir/placebo treatment and have an additional laboratory evaluation done within 1 week to confirm these results. If the additional evaluation confirms the elevated bilirubin and ALT, the patient discontinue all study treatments i.e. alisporivir/placebo, peg-IFNα2a and RBV and continue the study as scheduled.

Patients with total bilirubin levels >5×ULN interrupt alisporivir/placebo treatment. Peg-IFNα2a and RBV treatment should not be interrupted because hyperbilirubinemia is not expected to be causally related to Peg-IFNα2a or RBV treatment.

The following monitoring plan is applied: Patients with total bilirubin levels >5×ULN have alisporivir treatment interruption for 1 week. Peg-IFNα2a and RBV treatment is not interrupted because of hyperbilirubinemia. At the next scheduled weekly visit or after the patient has been recalled after 1 week (if hyperbilirubinemia occurs after treatment week 2), further biochemistry tests is conducted to confirm the expected decline in total bilirubin levels.

If the total bilirubin level has decreased to ≦5×ULN, then the investigator instruct the patient to re-start alisporivir treatment and have a repeat test again after 1 week.

If at this second test, the total bilirubin level is >5×ULN, and the patient has stabile or improving ALT from baseline, then alisporivir treatment can be withheld for a maximum one week further.

At the end of the second week without alisporivir therapy, the next blood test is performed. If this test shows that total bilirubin is ≦5×ULN, the investigator instruct the patient to re-start alisporivir treatment (again, only if ALT is stable or improving).

A further test is performed 1 week later to confirm that the total bilirubin level is still ≦5×ULN.

The maximum duration without alisporivir treatment is 2 weeks, either as continuous interruption or 2 separate weeks.

1. A method for treating a patient infected with Hepatitis C virus, the method comprising
administering alisporivir in an amount of about 400 to about 600 mg twice per day.

2. A method according to claim 1 characterized in that alisporivir is to be administered in combination with standard of care or a direct antiviral agent.

3. A method according to claim 1 characterized in that alisporivir is to be administered for up to 24, 48 or 72 weeks.

4. A method according to claim 2, wherein the standard of care is a combination of interferon with ribavirin.

5. A method according to claim 4, wherein said interferon is pegylated interferon alpha-2a, and is administered in an amount of 180 micrograms once per week.

6. A method according to claim 2, wherein said direct antiviral agent is ANA598.

7. A combination of alisporivir, and standard of care or a direct acting antiviral agent for use in treatment of a Hepatitis C virus infected patient characterized in that alisporivir is to be administered in an amount of about 400 to about 800 mg twice per day for up to 24, 48 or 72 weeks.

8. A therapeutic regimen comprising administering alisporivir in an amount of about 400 to about 600 mg twice per day for up to 24, 48 or 72 weeks and wherein alisporivir is administered in combination with standard of care or a direct acting antiviralagent.

9. A pharmaceutical composition comprising alisporivir for use according to claim 1.

10. A package comprising the pharmaceutical composition according to claim 9 in combination with instructions to administer said composition.
